(19) United States
(12) Patent Application Publication
(10) Pub. No.: US 2007/0197576 A1
(43) Pub. Date: Aug. 23, 2007

(54) Production of Cabergoline and Novel Polymorphic Form Thereof

(75) Inventors: Alan Greenwood, Hitchin (GB); Parveen Bhatarah, Cranford (GB); Raza Mahmood, Luton (GB); Derek McHattie, Stotfold (GB)

Correspondence Address: RAKOCZY MOLINO MAZZOCHI SIWIK LLP, 6 W. HUBBARD ST., SUITE 500, CHICAGO, IL, 60610, US

(73) Assignee: Resolution Chemicals Limited, Stevenage (GB)

(21) Appl. No.: 11/672,402

(22) Filed: Feb. 7, 2007

Related U.S. Application Data
(60) Provisional application No. 60/872,423, filed on May 24, 2006.

(30) Foreign Application Priority Data
Feb. 8, 2006 (GB) 0602557.1

(51) Int. Cl. A61K 31/48; C07D 457/02

(52) U.S. Cl. 514/288; 546/67

(57) ABSTRACT

The present application relates to a novel polymorphic form of cabergoline comprising cabergoline and t-amyl methyl ether, designated Form TAME cabergoline, together with a novel method of producing cabergoline.

The present application relates to a novel polymorphic form of cabergoline. The invention further provides a novel method of producing cabergoline.

Cabergoline is an ergoline derivative with the systematic name 1-((6-allylergolin-8β-yl)-carbonyl)-1-(3-dimethylaminopropyl)-3-ethylurea and having the following formula (I).

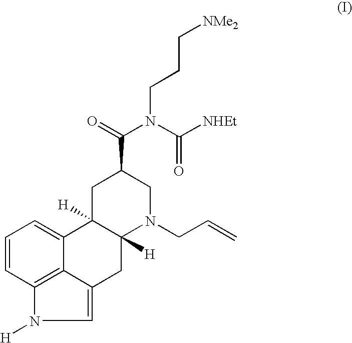

It is known for treatment of a number of diseases, including CNS disorders, reversible obstructive airways disease, prolactin inhibition, and for controlling intra-ocular pressure and for treating glaucoma.

In the final step of the synthetic pathway leading to cabergoline, an allylic acid intermediate of Formula (II) 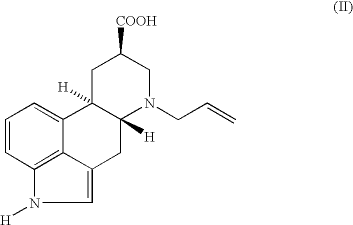
is reacted with N-(3-dimethylaminopropyl)-N-ethyl carbodiimide (EDAC) to produce cabergoline, i.e.:

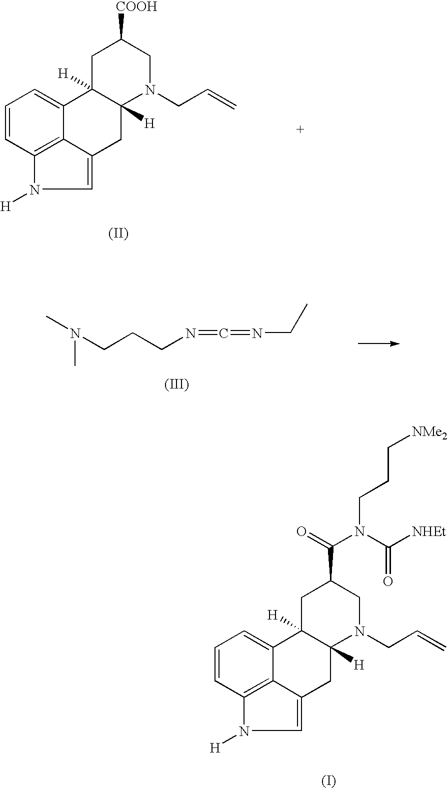

In a known procedure for producing cabergoline by this route, a mixture of the intermediate of formula (II), EDAC (III) and triethylamine are reacted in the presence of dimethylformamide and then extracted with dichloromethane (see, e.g. “Synthesis and nidation inhibitory activity of a new class of ergoline derivatives”, Brambilla, E. et al.; Eur. J. Chem. 24 (1989) 421-426.

The cabergoline so produced may then be subjected to purification procedures (including column chromatography and crystallisation from various solvents) and converted to a desired polymorph for incorporation into pharmaceutical preparations. Typical purification procedures include fractional crystallization from various solvents, as well as solvent extraction procedures. These are aimed at removing impurities, including an unwanted cabergoline isomer of Formula (IV), as well as producing a product consisting essentially of a desired cabergoline polymorph.

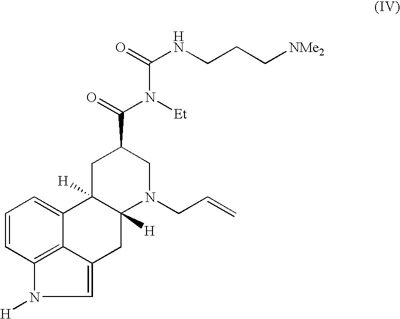

Additionally, the procedures are often designed to produce a stable, pure form of cabergoline which is suitable for storage prior to being converted to a desired polymorph and/or formulated with excipients etc.

A number of different forms of cabergoline are known and, by way of example, WO 01/72747 describes Form II cabergoline and WO 01/72746 describes Form VII cabergoline.

Form I cabergoline is of particular interest, and its preparation is described in WO 01/70740, WO 03/078392 and WO 03/078433. It is known from WO 01/70740 to prepare crystalline Form I cabergoline from a solvent comprising a toluene/diethyl ether mixture. From WO 03/078392 and WO 03/078433 it is known to prepare a solvate of cabergoline and toluene, and to obtain crystalline Form I cabergoline by drying the solvate.

Our WO 05/105796 describes and claims a process for producing Form I cabergoline in high yield and purity and with desirable particle size distribution utilising ethylbenzene, optionally in conjunction with n-heptane, as solvent. WO 05/105796 further describes a cabergoline ethylbenzene solvate. Our UK Patent Application Nos. 0505965.4 and 0515430.7 describe and claim processes for producing Form 1 cabergoline in high yield and purity and with desirable particle size distribution utilising 4-fluorotoluene, 1-chloro-4-fluorobenzene, 1,4-difluorobenzene or 1,3,5-trimethylbenzene as solvent, again, optionally in conjunction with n-heptane.

A series of polymorphs of cabergoline are also described in WO 2004/101510.

In more detail, in a typical procedure for producing cabergoline, the allylic acid of Formula (II) is reacted with EDAC in a suitable reaction medium. For example, dimethylformamide may be used as reaction medium (as in the procedure of Brambilla et al.) or as an alternative, acetonitrile may be used. After various solvent extraction steps, a solution of cabergoline is obtained, which may be used as starting material for further purification and for producing desired polymorphs for incorporation into pharmaceutical preparations, e.g. by the procedures of the above-mentioned International Patent Application No. WO 05/105796 and UK Patent Application Nos. 0505965.4 and 0515430.7.

It is often not necessary to purify the solution resulting from the above Reaction Scheme and to convert it to desired polymorphs straight away, and it may be convenient to store it for eventual use. Clearly, it is inconvenient to store a solution in view of its bulk and it is advantageous for the cabergoline to be stored in solid form. However, where the solution is a solution of cabergoline in acetonitrile, it is impractical to obtain cabergoline directly from the solution, because only an oil is obtainable by simply evaporating off the acetonitrile. Also, evaporation and other available techniques for obtaining solid cabergoline from the acetonitrile solution often results in an impure product being obtained in which the cabergoline may be contaminated with impurities, including cabergoline isomers, e.g. the isomer of Formula (IV) above.

The present invention derives from research into the use of various solvents in the production of cabergoline and its polymorphs and solvates and especially addresses the problem of producing a novel form of cabergoline which has exceptionally high chemical and polymorphic purity, and has properties that make it especially useful for storing in bulk.

In the research program leading to the present invention, the synthetic procedure described above was modified by using trifluoromethyl benzene (BTF) as, or as a component of the reaction medium for the reaction of the intermediate of formula (II) and EDAC. It was found that by using BTF, the proportion of undesired cabergoline isomers was lower than when dimethylformamide is used (as per the procedure of Brambilla et al.) and that the environmental problems of using dichloromethane as extracting solvent could be avoided. Further, the cabergoline resulting from the process was found to contain a lower proportion of unwanted isomers (including the isomer of formula (IV)) than in either of the prior processes referred to, i.e. the process of Bramilla et al. using dimethylformamide as reaction medium, and processes using acetonitrile.

In a further aspect of the invention, t-amyl methyl ether (TAME) was used as extracting solvent for the purification of relatively impure cabergoline. It was surprisingly found that a t-amyl methyl ether solvate could be readily isolated directly in a high state of purity and that this solvate could, if desired, be stored in preparation for transforming it into desired cabergoline polymorphs. It was also found that TAME was also useful in preparing pure, polymorphically homogeneous cabergoline from already partially purified product.

Thus, the present invention, according to one aspect thereof, provides a method of preparation of a novel solid form of cabergoline having a high chemical and polymorphic purity which comprises forming a cabergoline solution in a solvent comprising t-amyl methyl ether and recovering said solid form from the solution.

The present invention further provides a novel t-amyl methyl ether solvate of cabergoline. Said solvate is distinct from known polymorphs of cabergoline and is designated herein as “Form TAME cabergoline”.

According to a further aspect of the present invention, there is provided Form TAME cabergoline, which exhibits an X-ray diffraction pattern comprising peaks expressed in degrees two-theta, at approximately 13.99, 15.63, 16.16, 16.68, 17.06, 17.78, 20.78, 21.68, 23.40, 23.48 and 25.88.

According to another aspect of the present invention, there is provided Form TAME cabergoline, which exhibits an X-ray diffraction pattern of Table 1.

Also provided by the present invention is Form TAME cabergoline, which exhibits an X-ray powder diffraction pattern substantially the same as shown in FIG. 4b.

Accordingly, the present invention provides cabergoline Form TAME comprising less than 2 wt % of other polymorphs.

In one aspect of the invention, the cabergoline Form TAME comprises less than 1 wt % of other polymorphs.

In another aspect of the invention, the cabergoline Form TAME comprises less than 0.5 wt % of other polymorphs.

In a further aspect of the invention, the cabergoline Form TAME comprises less than 0.1 wt % of other polymorphs.

In a method of the invention set out in more detail in the examples, cabergoline may be dissolved in a solvent which comprises t-amyl methyl ether, and the solution cooled to a temperature of 5° C. or below. According to the first aspect of the invention, the solvent preferably comprises at least 75% by volume of t-amyl methyl ether, preferably at least 95%, and more preferably, at least 98%. In a specific embodiment the solvent consists solely of t-amyl methyl ether.

Alternatively, for example as part of the preparative procedure for synthesising cabergoline, t-amyl methyl ether may be used as a solvent for extracting cabergoline from an aqueous phase. Thus, according to a further aspect of the present invention, there is provided a method of preparation of a novel solid form of cabergoline having a high chemical and polymorphic purity which comprises extracting cabergoline from an aqueous solution thereof using a solvent comprising t-amyl methyl ether and recovering said solid form from the t-amyl methyl ether phase.

In one aspect of the invention, the solvent comprises at least 75% by volume of t-amyl methyl ether, more preferably at least 95%, and yet more preferably, at least 98%. In a specific embodiment the solvent consists solely of t-amyl methyl ether.

In embodiments of the invention set out in more detail in the examples below, cabergoline is dissolved in a solvent consisting of t-amyl methyl ether. This is conveniently done above room temperature, typically about 30 to 60° C., preferably about 40 to 50° C. and the resulting solution is preferably filtered to remove particulate material. The temperature of the solution is then lowered to 20 to 30° C. or below, preferably 26 to 28° C. and a precipitate of cabergoline/t-amyl methyl ether solvate formed. This can be encouraged by stirring and also by seeding, for example using crystalline Form I cabergoline.

The resulting suspension may then conveniently be cooled further, for example to 0 to 5° C. and held at this temperature for a period of 10 to 20 hours.

The resulting slurry may then be filtered to recover solid, which is optionally washed, for example with small quantities of t-amyl methyl ether, and then dried to yield cabergoline/t-amyl methyl ether solvate in high purity. The product may be optionally dried under vacuum or in an inert gas atmosphere. The product has been determined to be a novel polymorph, which we have designated “cabergoline Form TAME”.

The cabergoline Form TAME can be utilised further in the production of other forms of cabergoline with the advantage that the method results in cabergoline having a high purity. The present invention accordingly provides a method of preparing cabergoline Form I, which comprises converting cabergoline Form TAME into cabergoline Form I.

In one aspect of the invention, the cabergoline Form TAME is dried to remove the t-amyl methyl ether solvent and the cabergoline produced is then converted into Form I cabergoline.

In another aspect of the invention, the cabergoline Form TAME, or the cabergoline produced by drying thereof, is dissolved in a solvent comprising toluene, ethylbenzene, 4-fluorotoluene, 1-chloro-4-fluorobenzene, 1,4-difluorobenzene, 1,3,5-trimethylbenzene or xylene and cabergoline Form I is recovered from the solution formed.

According to a further aspect of the present invention, there is provided a method of producing cabergoline, which comprises reacting a compound of formula (II): 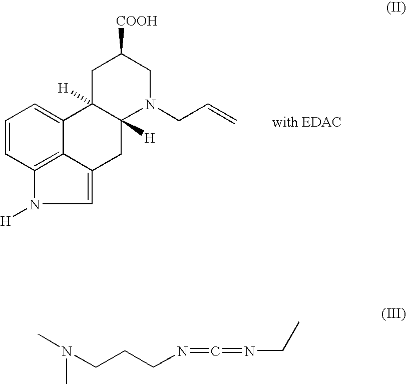
in a solvent, which comprises trifluoromethylbenzene.

In one aspect of the invention, the N-(3-dimethylaminopropyl)-N-ethyl carbodiimide hydrochloride (EDAC.HCl) is combined with trifluoromethylbenzene (BTF) and aqueous alkali, and a solution of EDAC in BTF is recovered.

In another aspect of the invention, the solution of EDAC in BTF is combined with a suspension of the compound of formula (II) in BTF.

In a further aspect of the invention, the reaction mixture is heated to a temperature of 35 to 38° C.

The invention will now be described in more detail in the following Examples and with reference to the accompanying drawings.

### BRIEF DESCRIPTION OF THE FIGURES

FIGS, 1a(1), 1a(2), 1b(1) and 1b(2) show ¹³CPMAS spectra of the product of Example 2 (designated Form TAME cabergoline) at −30° C. and at ambient temperature, respectively;

FIG. 2 shows a differential scanning calorimetry (DSC) trace of Form TAME cabergoline;

FIG. 3 shows a DRIFT IR scan of Form TAME cabergoline;

FIGS. 4a and 4b show X-ray diffraction patterns of Form TAME cabergoline;

FIGS. 5a(1), 5a(2), 5b(1) and 5b(2) show the results of particle size determinations for Form TAME cabergoline.

The present invention is described in further detail in the following non-limiting examples. The great variety of options falling within the scope of the invention will be readily determinable by those skilled in the art upon consideration of the general method described above and exemplified below.

### EXAMPLES

### Example 1

### Synthesis of Cabergoline

N-(3-Dimethylaminopropyl)-N-ethyl carbodiimide hydrochloride (EDAC.HCl) is added to a stirred mixture of trifluoromethylbenzene (BTF) and 25% w/w aqueous potassium carbonate. The mixture is stirred until a clear two-phase solution is obtained. The layers are allowed to separate and the lower aqueous layer is discarded. The upper organic layer is stirred with anhydrous potassium carbonate and filtered to provide a solution of EDAC in BTF.

A suspension of a compound of formula (II) in BTF is stirred at IS to 24° C. and the required quantity of EDAC in BTF solution is charged. The resulting suspension is then heated to a temperature of 35 to 38° C. and maintained at this temperature until the reaction is complete. The solution is filtered and purified water added. Glacial acetic acid is then added to bring the pH to 5.0 to 5.5. The upper aqueous phase is separated. t-Butyl methyl ether is added to the upper aqueous phase and a 20% w/w potassium hydroxide solution is added to adjust the mixture to pH 9.5 to 10.0.

The layers are separated and the lower aqueous layer is extracted with t-butyl methyl ether. The two upper organic layers are combined and washed with 13% aqueous sodium chloride. The upper organic layer is then separated and stirred with charcoal. The mixture is then filtered and concentrated under vacuum at 35 to 38° C. to about 2 to 3 volumes. Acetonitrile is added and the solvent exchanged via distillation under vacuum at 35 to 38° C. to about 2 to 3 volumes.

The resulting acetonitrile solution may then be used as starting material for producing desired polymorphs for incorporation into pharmaceutical preparations, e.g. by the procedures of the above-mentioned international Patent Application No. WO 05/105796 and UK Patent Application Nos. 0505965.4 and 0515430.7.

### Example 2

### Formation of TAME Solvate

Cabergoline (4.0 g) was dissolved in 10 ml of t-amyl methyl ether and placed on a heating mantle set for 50° C. A clear solution was obtained after 15 minutes when the temperature had reached 41° C. The solution was filtered through a 0.45 micron filter and the resulting solution cooled to 20 to 26° C. and seeded with 1% w/w of pure Form I cabergoline.

Crystallisation commenced at 27° C. and the resulting suspension was cooled to 0 to 5° C. and held at this temperature for over 16.5 hours. The resultant white solid was filtered under an atmosphere of nitrogen. The obtained product had a damp weight of 3.55 g, corresponding to a recovery of 88.8%.

Samples of the product were subjected to ¹³C mass spectrometry, differential scanning calorimetry (DSC), DRIFT IR, DSC, X-ray crystallographic analysis, gas chromatography, HPLC and particle size analysis and determined to be of a new crystalline form. Further the product was found to be of exceptionally high purity and to be free of the cabergoline isomer of formula (IV).

### RESULTS OF ANALYSES

1. ¹³C CPMAS Spectra

Two samples of the product of Example 2 were subjected to ¹³C CPMAS spectroscopy at −30° C. and at ambient temperature. The results are shown in FIGS. 1a and 1b respectively.

The samples were found to be exceptionally pure and there was no evidence of the presence of any isomers or other polymorphic forms. Narrow lines (labelled “s” in the ambient trace of FIG. 1b are believed to derive from solvent (t-amyl methyl ether).

Intensity differences between the −30° C. and ambient temperature traces (notably at 32 and 43.2 ppm) are considered to be due to a change in the motion of the cabergoline NMe₂ side-chain.

2. X-ray Crystallographic Analysis

X-ray crystallographic analysis indicated that the product of Example 2 consisted of a single polymorph.

Experimental

X-ray powder diffraction data were collected at room temperature on an automated Philips PW 1050/30 X-ray diffractometer, using Ni filtered CuKα radiation (λ=1.5418 A), in flat plate θ/2θ geometry. Data were collected in the range 5 to 70° 2 θ, in steps of 0.05°, with a scan time of 2s per step and a 1s delay time. The sample was stored at −20° C. prior to the experiment.

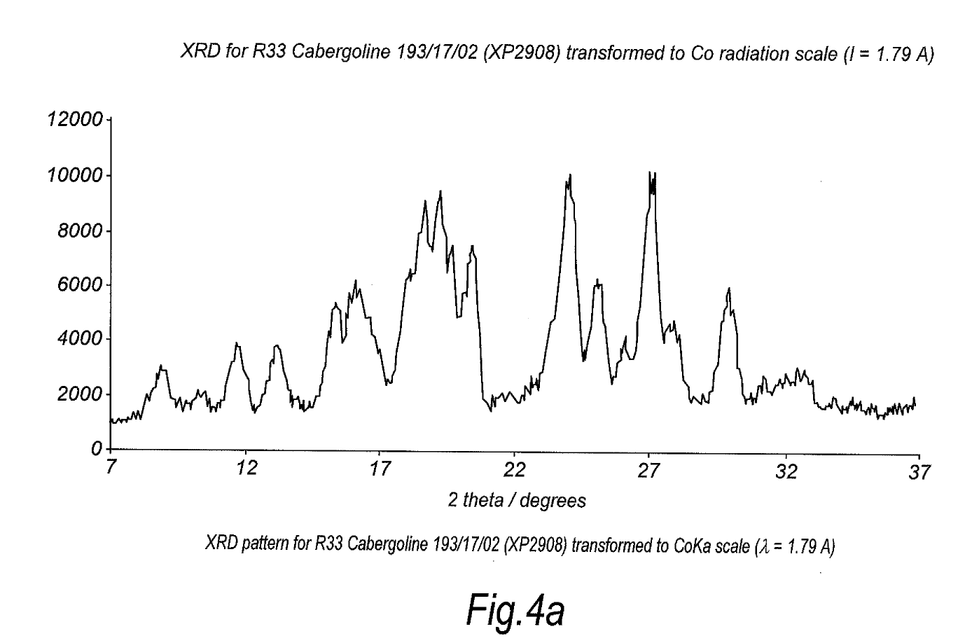

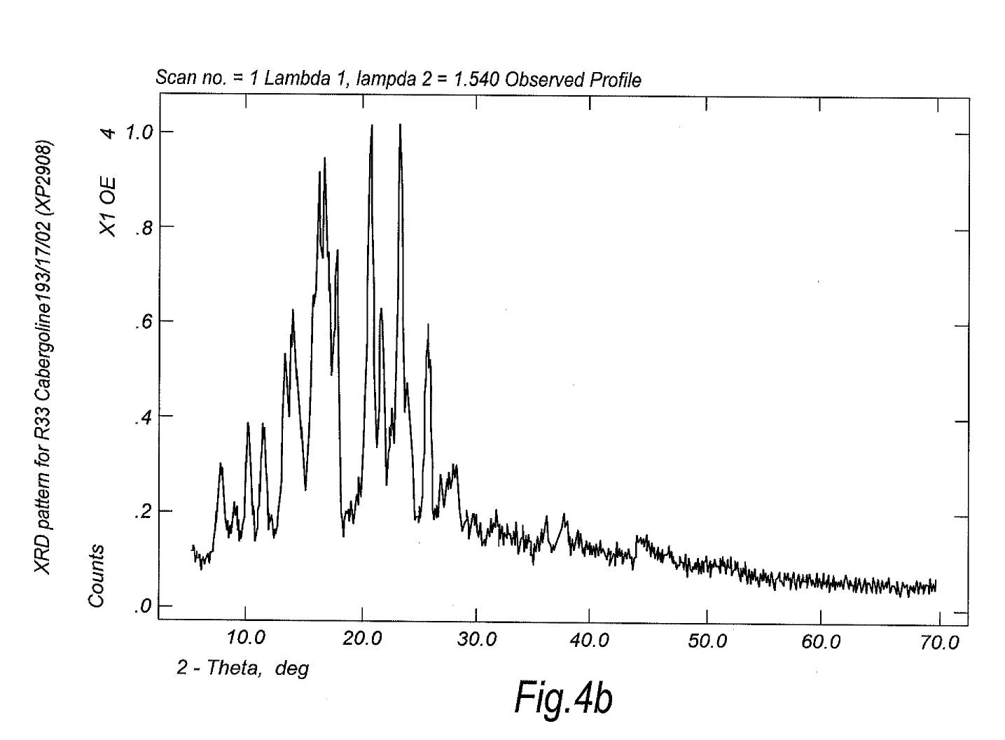

The X-ray powder diffraction pattern for the sample examined is shown in FIG. 4a and 4b, with measured peak data in Table 1.

The XRD pattern is strong, but showed peak broadening. This broadening is a consequence of small particle size and reflects relatively low crystallinity. The observed data were compared to the diffraction patterns for the known polymorphs and solvated forms. The data do not correspond to Form I, Form II or solvated Form V. The data were transformed to compare with those presented in patent application PCT/US2004/014367 collected using CoKα radiation. No clear match is evident.

TABLE 1

| 2θ/° | d/Å | % |
|---|---|---|
| 7.729 | 11.439 | 21 |
| 8.974 | 9.853 | 13 |
| 10.133 | 8.729 | 32 |
| 11.437 | 7.737 | 32 |
| 13.292 | 6.661 | 47 |
| 13.987 | 6.332 | 57 |
| 15.63 | 5.666 | 61 |
| 16.160 | 5.485 | 89 |
| 16.681 | 5.314 | 92 |
| 17.055 | 5.199 | 71 |
| 17.775 | 4.990 | 73 |
| 20.776 | 4.275 | 99 |
| 21.684 | 4.098 | 58 |
| 22.572 | 3.939 | 36 |
| 23.403 | 3.801 | 100 |
| 23.480 | 3.789 | 100 |
| 24.137 | 3.687 | 42 |
| 25.875 | 3.443 | 55 |
| 26.938 | 3.310 | 22 |
| 27.646 | 3.227 | 22 |
| 28.226 | 3.162 | 24 |

3. Gas Chromatography

The product of Example 2 was subjected to gas chromatography using the following apparatus and conditions.

Apparatus

GC system consisting of:

Regulated Helium, Nitrogen and air gas carrier

Automated Injector

Thermostated column oven, injector port and FID detector

Reagents

Tert-amyl methyl ether

HPLC grade Dimethylformamide (DMF) (Dissolving solvent)

Chromatoqraphic Conditions

Column: ZB-624 (30M×0.32 MM×1.8 μM) with 1 m deactivated silica retention gap.

Inlet: 250° C., Split 40:1. He column head pressure 0.06 MPa (9.0 psi), constant pressure mode.

Injection Volume: 5.0 μL.

Oven: 85° C. (5 min hold) then to 250° C. at 120° C./min hold for 5.0 minutes.

Detector: FID at 250° C. N2 make-up at 45 ml/min, H2 at 40 ml/min and air at 450 ml/min.

Approximate Retention Times

Tert-amyl methyl ether 4.84 minutes

Standard preparation from 0.0 mg/mL to 2.724 mg/mL

When plotted against concentration, the peak areas showed a high linearity. The sample contained 18.75% TAME.

4. DSC, DRIFT IR, Particle Size Analysis

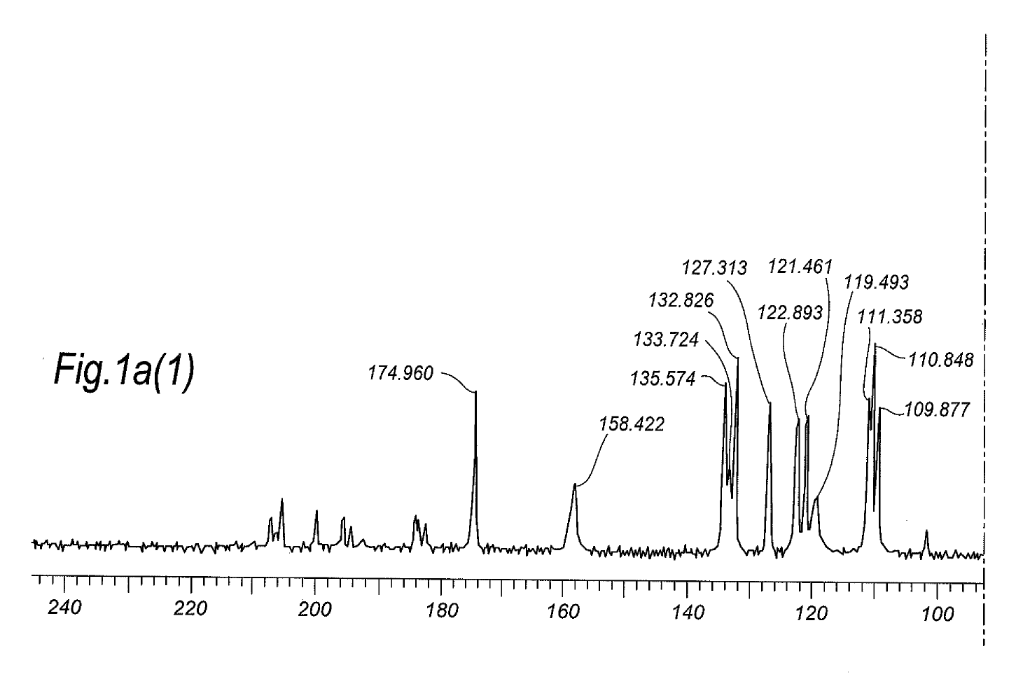

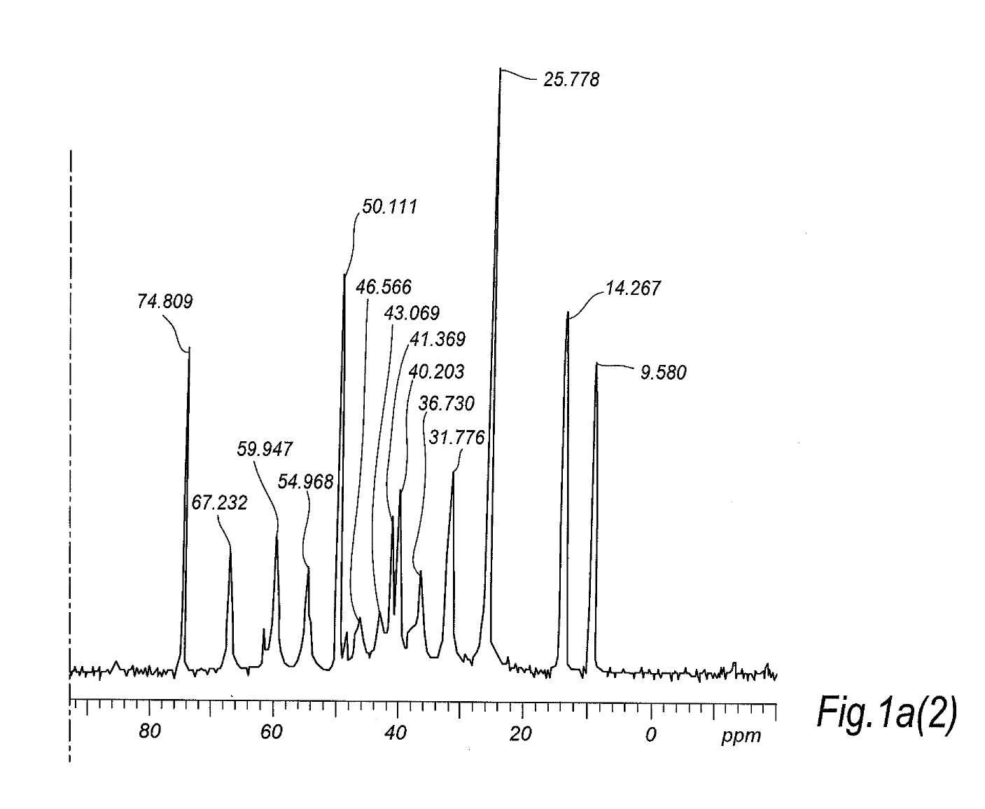

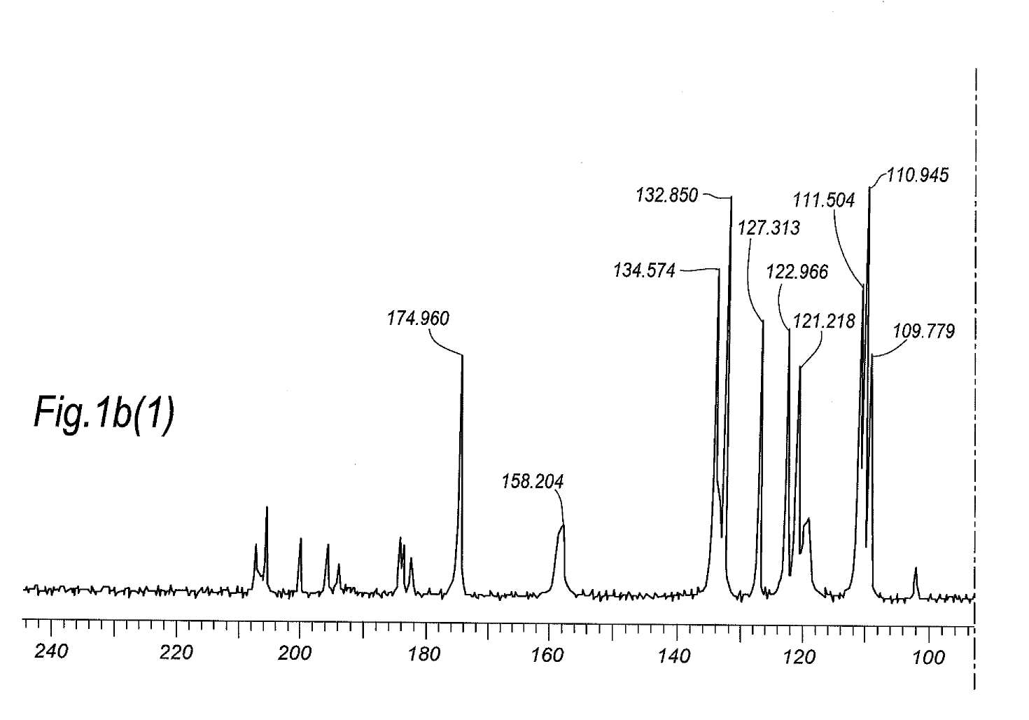

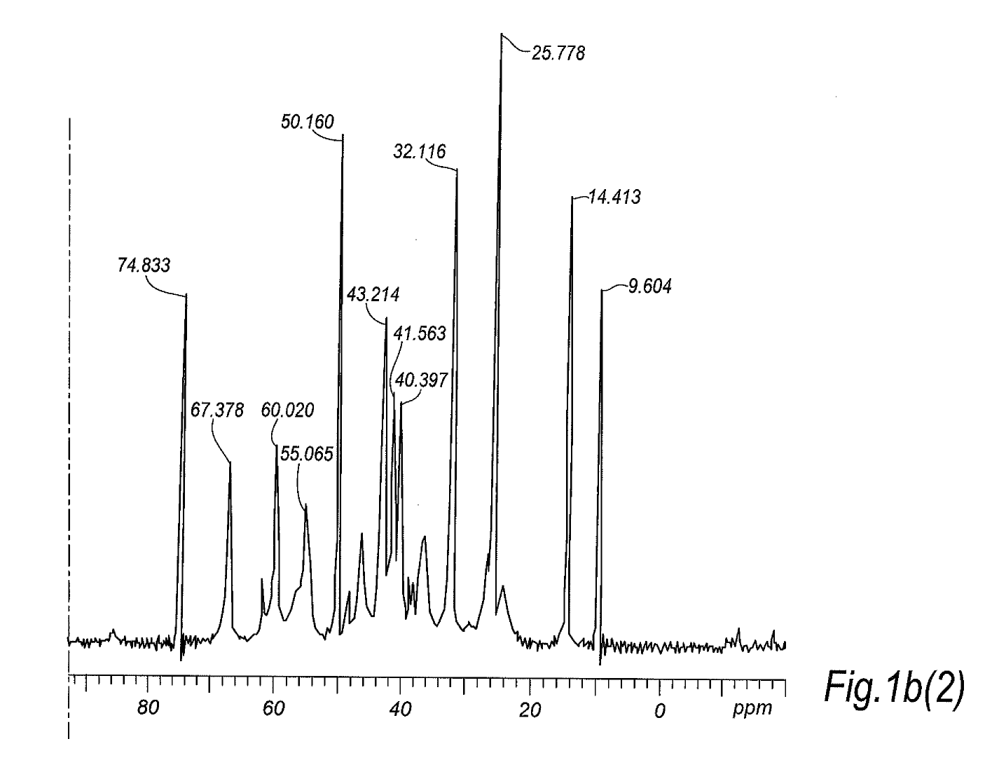

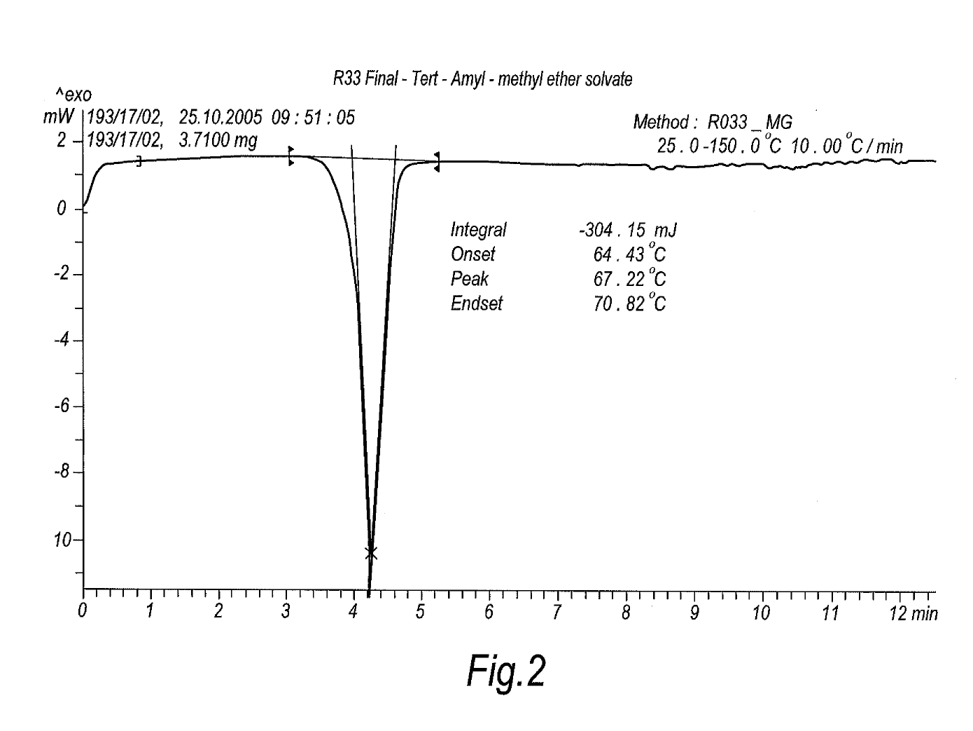

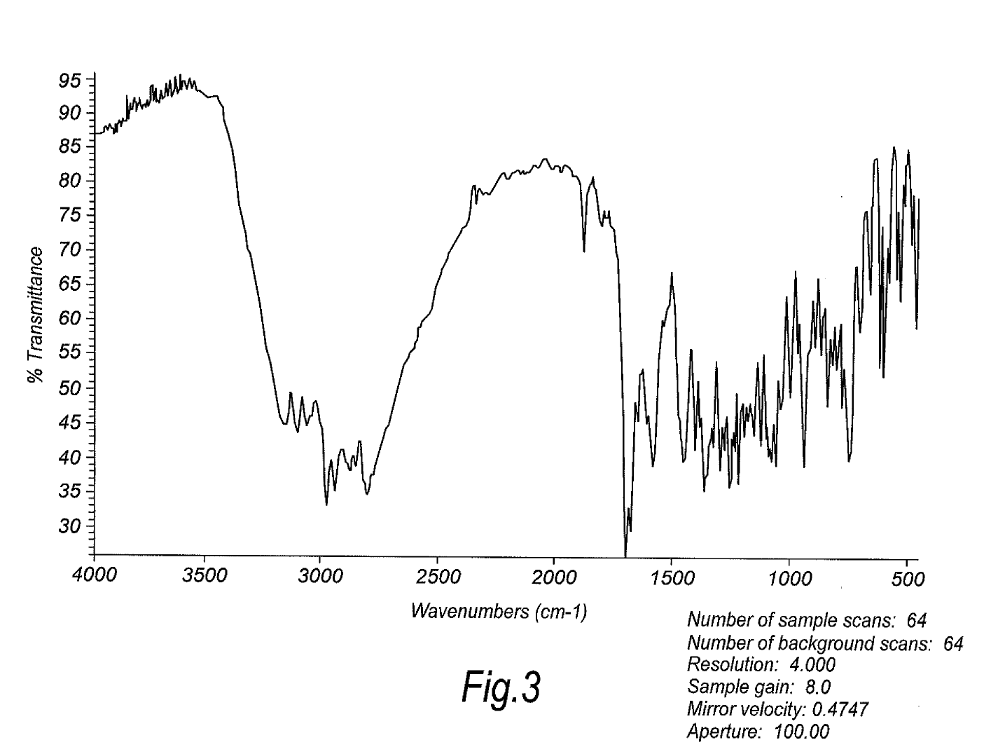

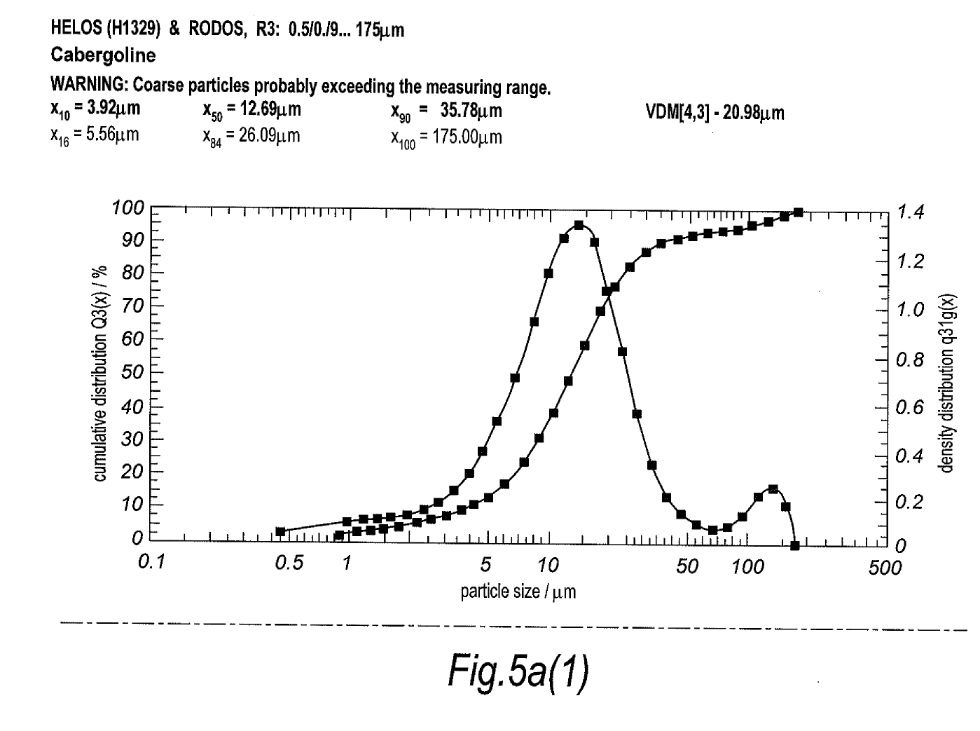

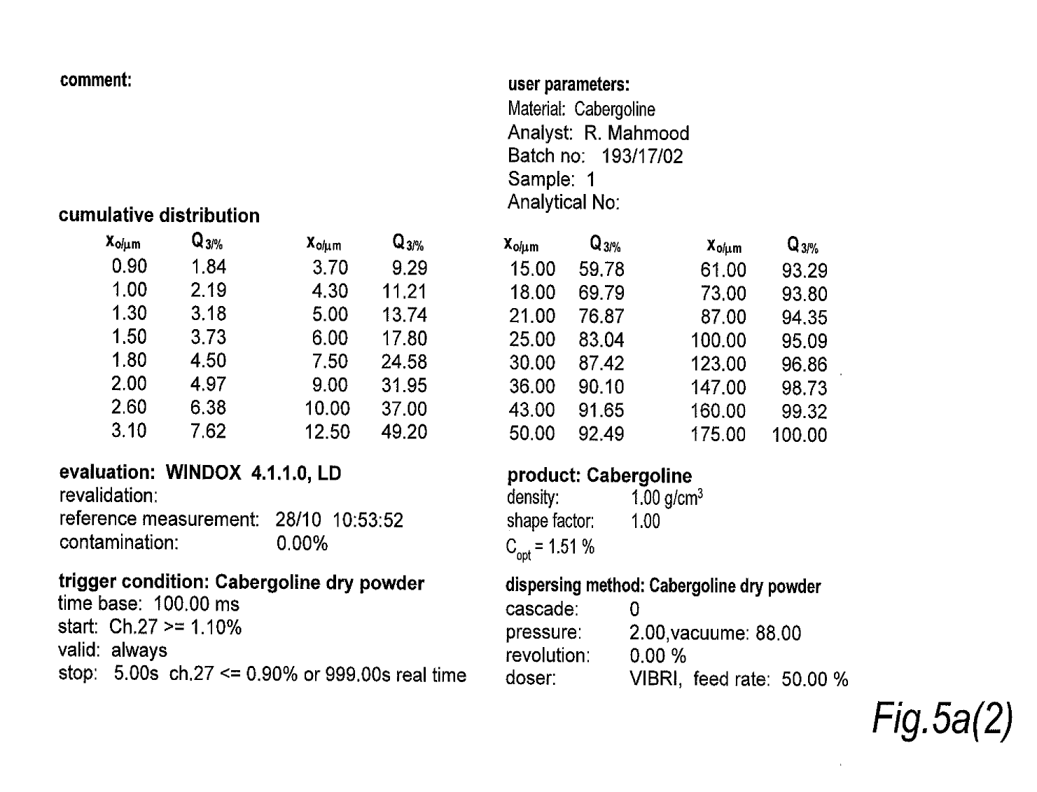

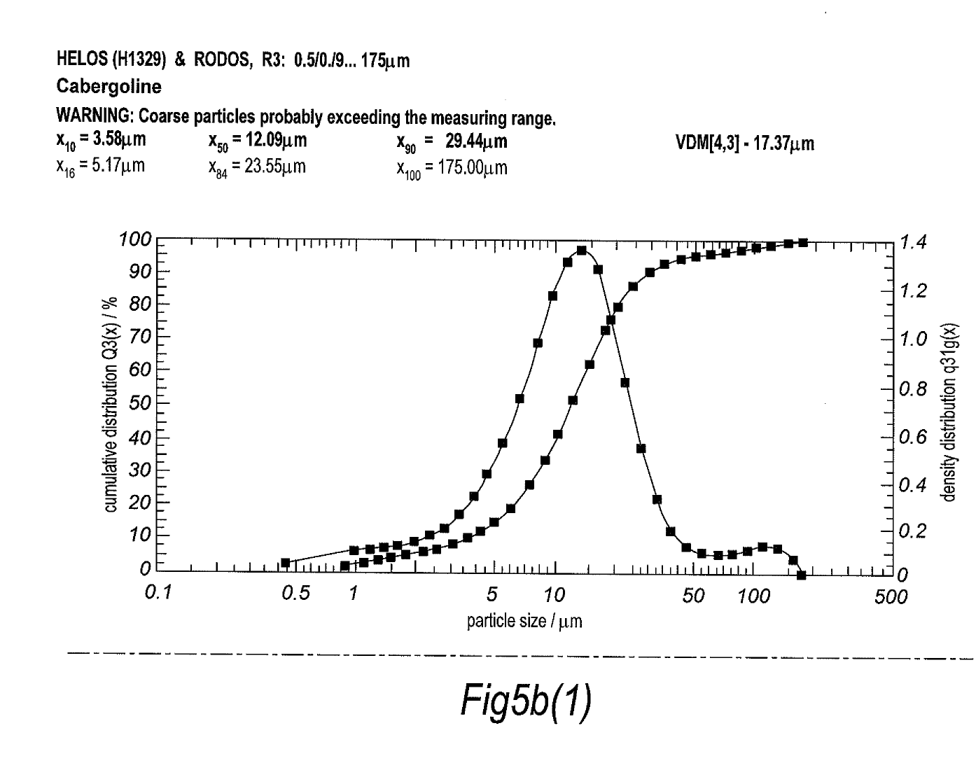

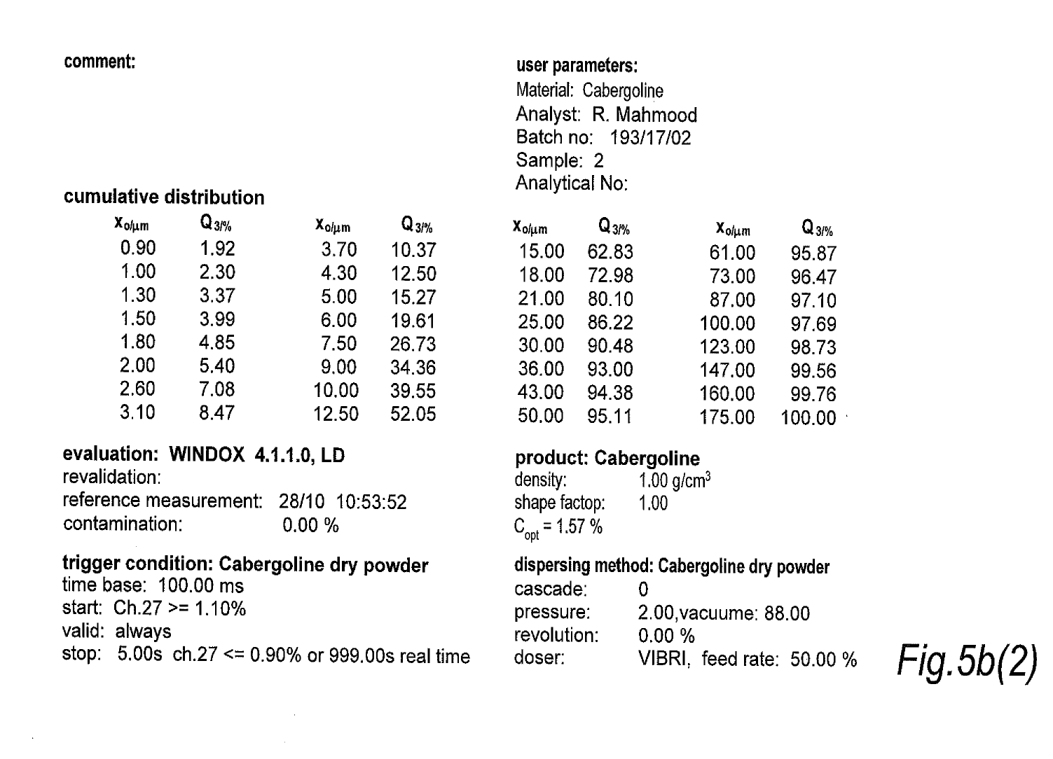

The data in FIGS. 2, 3, 5a and 5b confirm the purity and excellent particle size distribution of cabergoline Form TAME. HPLC analysis showed the material to have a purity of 99.8%.

While various embodiments of the present invention have been described above, it should be understood that such disclosures have been presented by way of example only, and are not limiting. Thus, the breadth and scope of the present invention should not be limited by any of the above-described exemplary embodiments, but should be defined only in accordance with the following claims and their equivalents.

Having now fully described the invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference in their entirety.

1. A solvate of cabergoline comprising cabergoline and t-amyl methyl ether, designated Form TAME cabergoline.

2. The cabergoline Form TAME of claim 1, which exhibits an X-ray diffraction pattern comprising peaks expressed in degrees two-theta at approximately 13.99, 15.63, 16.16, 16.68, 17.06, 17.78, 20.78, 21.68, 23.40, 23.48 and 25.88.

3. The cabergoline Form TAME of claim 2 having the X-ray powder diffraction pattern substantially as shown in FIG. 4b.

4. The cabergoline Form TAME of claim 1 comprising less than 2 wt % of other polymorphs.

5. The cabergoline Form TAME of claim 4 comprising less than 1 wt % of other polymorphs.

6. The cabergoline Form TAME of claim 5 comprising less than 0.5 wt % of other polymorphs.

7. The cabergoline Form TAME of claim 6 comprising less than 0.1 wt % of other polymorphs.

8. A method of preparing the cabergoline Form TAME of claim 1, which comprises dissolving cabergoline in a solvent comprising t-amyl methyl ether and recovering cabergoline Form TAME from the solution formed.

9. The method of claim 8, wherein the solution is cooled to a temperature of 5° C. or below.

10. The method of claim 9, wherein the solution is cooled to a temperature of 0 to 5° C.

11. The method of claim 8, wherein the solvent comprises at least 75% by volume of t-amyl methyl ether.

12. The method of claim 11, wherein the solvent comprises at least 95% by volume of t-amyl methyl ether.

13. The method of claim 12, wherein the solvent comprises at least 98% by volume of t-amyl methyl ether.

14. The method of claim 8, wherein the cabergoline Form TAME is recovered by filtration.

15. The method of claim 8, wherein the recovered cabergoline Form TAME is dried.

16. The method of claim 15, wherein the recovered cabergoline Form TAME is dried in an inert gas atmosphere.

17. The method of claim 15, wherein the recovered cabergoline Form TAME is dried under vacuum.

18. A method of preparing cabergoline, which comprises reacting a compound of formula (II) 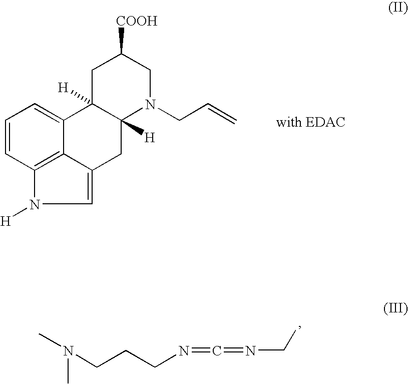
in a solvent, which comprises trifluoromethylbenzene.

19. The method of claim 18, wherein N-(3-dimethylaminopropyl)-N-ethyl carbodiimide hydrochloride (EDAC.HCl) is combined with trifluoromethylbenzene (BTF) and aqueous alkali, and a solution of EDAC in BTF is recovered.

20. The method of claim 19, wherein the solution of EDAC in BTF is combined with a suspension of the compound of formula (II) in BTF.

21. A method for purifying cabergoline, which comprises extracting cabergoline from an aqueous solution utilising a solvent, which comprises t-amyl methyl ether.

22. The method of claim 21 wherein the aqueous solution of cabergoline is the result of an aqueous work-up of the reaction of claim 18.

23. A method of preparing Form I cabergoline, which comprises converting cabergoline Form TAME into cabergoline Form I.

24. The method of claim 23, wherein the cabergoline Form TAME is dried to remove the t-amyl methyl ether solvent and the cabergoline produced is then converted into cabergoline Form I.

25. The method of claim 23, wherein the cabergoline Form TAME is dissolved in a solvent comprising toluene, ethylbenzene, 4-fluorotoluene, 1-chloro-4-fluorobenzene, 1,4-difluorobenzene, 1,3,5-trimethylbenzene or xylene and Form I cabergoline is recovered from the solution formed.

26. The method of claim 24, wherein the cabergoline produced is dissolved in a solvent comprising toluene, ethylbenzene, 4-fluorotoluene, 1-chloro-4-fluorobenzene, 1,4-difluorobenzene, 1,3,5-trimethylbenzene or xylene and Form I cabergoline is recovered from the solution formed.
